(19) United States
(12) Patent Application Publication
(10) Pub. No.: US 2026/0076753 A1
(43) Pub. Date: Mar. 19, 2026

(54) METHODS AND SYSTEMS FOR ACCESSING ANATOMICAL SPACES

(71) Applicant: THE JOHNS HOPKINS UNIVERSITY, Baltimore, MD (US)

(72) Inventors: Mandeep SINGH, Baltimore, MD (US); Jin Ung KANG, Baltimore, MD (US); Shoujing GUO, Baltimore, MD (US)

(73) Assignee: THE JOHNS HOPKINS UNIVERSITY, Baltimore, MD (US)

(21) Appl. No.: 19/400,713

(22) Filed: Nov. 25, 2025

Related U.S. Application Data
(63) Continuation of application No. PCT/US2024/031927, filed on May 31, 2024.
(60) Provisional application No. 63/470,016, filed on May 31, 2023.

(51) Int. Cl. A61B 34/20; A61B 5/00; A61B 17/34; A61F 9/007

(52) U.S. Cl. CPC A61B 34/20; A61B 5/0073; A61B 17/3403; A61F 9/00736; A61B 2034/2057

(57) ABSTRACT

The present disclosure relates to methods for performing minimally invasive anatomical space access, e.g., minimally invasive subretinal access (MISA), in connection with the delivery of therapeutic modalities to an anatomical space of interest, e.g., the subretinal space, and system components adapted to facilitate such delivery.

### CROSS-REFERENCE TO RELATED APPLICATIONS

This application is a continuation of International Patent Application No. PCT/US2024/031927, filed May 31, 2024, which claims priority to U.S. Provisional Application No. 63/470,016 filed May 31, 2023, the contents of which are incorporated by reference in their entireties, and to each of which priority is claimed.

### FIELD OF INVENTION

The present disclosure relates to methods for performing minimally invasive anatomical space access, e.g., minimally invasive subretinal access (MISA), in connection with the delivery of therapeutic modalities to an anatomical space of interest, e.g., the subretinal space, and system components adapted to facilitate such delivery.

### BACKGROUND

Acquired or inherited degenerative retinal diseases, including non-exudative age-related macular degeneration (AMD), retinitis pigmentosa (RP), and Stargardt macular dystrophy, are major global causes of blindness. The cells that degenerate in these conditions are the photoreceptor and retinal pigment epithelium (RPE) cells. Electronic, stem cell, and gene therapy modalities are in development, aiming to preserve, restore, or replace the structure and/or function of the photoreceptor and RPE cell layers. These modalities are delivered to the anatomical space known as the subretinal space.

Conventionally, the transvitreal ab interno approach has been used to gain access to the subretinal space. The typical key steps of this approach are removing the vitreous gel (i.e., vitrectomy) and inducing a perforation in the retina (i.e., retinotomy). Complications of ab interno access have included cataract formation, retinal detachment, intraocular hemorrhage, proliferative vitreoretinopathy (PVR), macular edema, and epiretinal membrane (ERM) formation all of which can cause vision loss. Another important risk of ab interno access is the potential for the retrograde reflux of therapeutic agents into the vitreous cavity via the retinotomy, thus reducing the subretinal therapeutic dose and promoting antigen exposure that may heighten the risk of immune rejection. Taken together, delivery-related complications of the ab interno approach have the potential to reduce efficacy, trigger adverse events, and threaten potential visual gains, thus compromising treatment outcomes. The vitrectomy and retinotomy steps are the main sources of the surgical morbidity of the ab interno approach.

In view of the foregoing, there remains an unmet need to develop strategies for subretinal delivery that minimize surgical morbidity by avoiding vitrectomy and retinotomy. More broadly, however, there also remains an unmet need in the art to develop strategies to access anatomical spaces more generally, particularly in a minimally invasive manner. For example, there remains a need for the development of strategies to access and deliver therapeutic modalities to anatomical spaces of interest, including, but not limited to, spinal cord spaces, intracranial spaces, e.g., ventricle spaces, subarachnoid spaces, submeningeal spaces, inner ear spaces, nasopharynx spaces, intra-abdominal spaces, intradermal spaces, intra-articular spaces, lung/pleural spaces, intracardiac spaces, and pancreatic/ gastrointestinal spaces, particularly in a minimally invasive manner.

### SUMMARY OF THE INVENTION

In certain embodiments, the present disclosure is directed to image-guided surgery (IGS) approaches to access a cavity or natural cleavage plane in the human body using fiber-optic distal sensor-based imaging, e.g., optical coherence tomography (OCT)-based imaging. In certain embodiments, the present disclosure is directed to a coaxial guide device comprising: an adapter configured to contact a surface at least partially encapsulating, directly or indirectly, an anatomical space (i.e., a cavity or natural cleavage plane in the human body); and a needle drive comprising a needle disposed in a needle guide. In certain embodiments, the adaptor comprises a vacuum channel. In certain embodiments, the adapter comprises an imaging component. In certain embodiments, the imaging component is stabilized via vacuum suction of the vacuum channel contacting the surface of the anatomical space. In certain embodiments, the needle drive comprises a rotation handle and pitch threads operably connected to the needle guide. In certain embodiments, rotation of the rotation handle advances the needle disposed in the needle guide. In certain embodiments, the imaging component comprises an optical coherence tomography sensor for visualization of an entry point of the needle, such that a user can determine depth of penetration of the needle at entry. In certain embodiments, the optical coherence tomography sensor is positioned within the lumen of the needle. In certain embodiments, the anatomical space is selected from: a spinal cord space; an intracranial space, e.g., a ventricle space; a subarachnoid space; a submeningeal space; an inner ear space; a nasopharynx space; an intra-articular space; a lung/pleural space; an intracardiac, and a pancreatic/gastrointestinal space.

In certain embodiments, the present disclosure is directed to methods of delivering material to an anatomical space of interest comprising: visualizing the anatomical space of interest using an optical sensor stabilized by a coaxial guide device comprising a vacuum channel; accessing the anatomical space of interest using a needle disposed in the coaxial guide device; and depositing material in the subretinal space using a pair of thin, elongate strips of flexible material and thin layer of elastic material surrounding the pair of thin, elongate strips of flexible material form a flexible, expandable tube, wherein an elongate lumen extends between the pair of thin, elongate strips of flexible material for depositing the material.

In certain embodiments, the present disclosure is directed to a coaxial guide device for use in accessing the subretinal space comprising: an eye adapter comprising a vacuum channel; and a needle drive comprising a needle disposed in a needle guide. In certain embodiments, the vacuum channel is configured to contact a scleral surface. In certain embodiments, the eye adapter comprises an imaging component. In certain embodiments, the imaging component is stabilized via vacuum suction of the vacuum channel contacting the scleral surface. In certain embodiments, the needle drive comprises a rotation handle and pitch threads operably connected to the needle guide. In certain embodiments, rotation of the rotation handle advances the needle disposed in the needle guide. In certain embodiments, the imaging component comprises an optical coherence tomography sensor for visualization of an entry point of the needle, such that a user can determine depth of penetration of the needle at entry. In certain embodiments, the optical coherence tomography sensor is positioned within the lumen of the needle.

In certain embodiments, the present disclosure is directed to methods of delivering material to the retina comprising: visualizing the subretinal space using an optical sensor stabilized by a coaxial guide device comprising a vacuum channel; accessing the subretinal space using a needle disposed in the coaxial guide device; and depositing material in the subretinal space using a pair of thin, elongate strips of flexible material and thin layer of elastic material surrounding the pair of thin, elongate strips of flexible material form a flexible, expandable tube, wherein an elongate lumen extends between the pair of thin, elongate strips of flexible material for depositing the material. In certain embodiments, the optical sensor is an optical coherence tomography sensor. In certain embodiments, the optical sensor is stabilized by contacting the coaxial guide device comprising a vacuum channel to a scleral surface. In certain embodiments, accessing of the subretinal space via an incision in the sclera and choroid.

### BRIEF DESCRIPTION OF THE DRAWINGS

FIGS. 1A-1C. Schematic of the common-path swept source optical coherence tomography (CP-SSOCT) and coaxial guide (COG) components of the minimally invasive subretinal access (MISA) system. (1A) CP-SSOCT device diagram showing the balanced detector, swept source laser source, attenuator, and circulator connected to the sensor integrated needle. (1B) CP-SSOCT distal-sensor guided injection device with articulated arm supporting and stabilizing the translation stage, and (1C) COG device that enabled secure coupling the sensor integrated needle and driver to the scleral surface by vacuum suction.

FIGS. 2A-2B. Subretinal access cannula specifications. Top layer with specifications (2A). Composite view of the device with top and side views and photography of composite device (2B). The indentations in the top layer and the holes in the bottom layer, in combination with the rule measure marks on the while adapting individual variations in ocular anatomy

FIGS. 3A-3D. Ev vivo validation of common path swept source optical coherence tomography (CPSS-OCT) based detection of the subretinal plane from the ab externo trans-scleral direction. (3A) Experimental setup for ex vivo testing using cadaveric bovine eyes. (3B-3D) A-Scan images (right) and corresponding AB-mode images (left) of bovine retinal layers as needle advances toward it, (3B) ˜650 microns from choroid, (3C) needle touch on the external surface of the choroid, (3D) needle in the sub-retinal space. RPE: retinal pigment epithelium. IS/OS: inner segment/outer segment. ILM: Inner retinal membrane

FIGS. 4A-4C. In vivo validation of common path swept source optical coherence tomography (CPSS-OCT) based detection of the subretinal plane ab externo. (4A) Experimental setup of in vivo porcine eye surgery showing the CPSS-OCT guide device of FIG. 1C. A-scan images (right panels) and corresponding AB-mode images (left panels) of porcine retina as needle advances axially. (4B) ˜200 microns from choroid, (4C) needle in the sub-retinal space. RPE: retinal pigment epithelium. IS/OS: inner segment/outer segment

FIGS. 5A-5E. Concept and in vivo proof-of-concept of minimally invasive subretinal access (MISA) approach. The key steps of common path swept source optical coherence tomography (CPSS-OCT) guided transscleral subretinal access are shown with schematic concept diagram (top) and operative images (bottom). (5A) The sclera is dissected at the equator and a ring is marked to prepare the sclera for placement of the CP-SSOCT guide device. (5B) The CP-SSOCT guide device is secured on the sclera, the injection needle is advanced axially, and viscoelastic material is injected into the sub-retinal space (between the RPE and IS/OS A-scan peaks). (5C) The subretinal bleb is visualized. (5D) A sub-retinal delivery device is inserted into the bleb at the equator and advanced to the posterior retina. (5E) The sub-retinal delivery device is confirmed to be in the posterior sub-retinal space.

FIG. 6A-6D. Concept and in vivo proof-of-concept of minimally invasive subretinal access (MISA) approach. The key steps of common path swept source optical coherence tomography (CPSS-OCT) guided transscleral subretinal access are shown with schematic concept diagram (top) and operative images (bottom). (6A) The sclera is dissected at the equator and a ring is marked to prepare the sclera for placement of the CP-SSOCT guide device (white arrowhead denote scleral marking edges). (6B) The CP-SSOCT guide device is secured on the sclera, the injection needle is advanced axially, and subretinal space is identified on A-scan (between the RPE and IS/OS A-scan peaks). (6C) The subretinal bleb created by injection of viscoelastic material (top, asterisk—viscoelastic) (arrowhead—subretinal needle) and is visualized internally (bottom, asterisk—subretinal bleb) (arrow heads-bleb borders). (6D) A sub retinal delivery device (top and bottom, asterisk) is inserted into the bleb (top, hashtag—bleb) (top, arrow heads—sclerotomy edges) at the equator, advanced to the posterior retina and confirmed to be in the posterior sub retinal space (bottom, arrow-retinal vessels overlying posterior sub retinal delivery device) (bottom, hashtag-RPE displacement overlying sub retinal delivery device).

FIG. 7A-7D. Complications associated with minimally invasive subretinal access (MISA) approach. Retinal incarceration (7A, arrowhead) in the sclerotomy occurred in the first animals which was resolved by turning the infusion off prior to choroidotomy (7A). RPE displacement (7B, arrow heads) was observed where patches of RPE were seen superficial to the device (7B). The tip of the rounded tip of the SAC was noted to be embedded in the retina (white arrow, 7B). Subretinal (7C, arrowheads) and suprachoroidal (7D, arrowheads) hemorrhage was observed in a single case, presumably due to damage to choroidal vessels during SAC propagation.

FIG. 8A-8C. Subretinal access device (SAC) preliminary design and subsequent iteration. SAC geometry was too square and the sharp corners prohibited smooth insertion into the sclerotomy (8A). The SAC tip was angled and rounded to aid with smooth insertion (8B). Holes were added along the length of the SAC (top and bottom layers) to allow fixation to the sclera to stabilize the device during insertion (8C).

### DETAILED DESCRIPTION

The present disclosure relates to methods for performing minimally invasive anatomical space access, e.g., MISA, in connection with the delivery of therapeutic modalities to an anatomical space of interest, e.g., the subretinal space, and system components adapted to facilitate such delivery.

### Minimally Invasive Subretinal Access (MISA)

The subretinal space targeted by MISA-based delivery strategies is a potential space that is located between the photoreceptor and RPE cell layers that are normally adherent to one another. The actual subretinal space is created by the pathologic or iatrogenic accumulation of material between the photoreceptor and RPE cell layers. In various disease states, exudate or hemorrhage can accumulate in the subretinal space. In vitreoretinal surgery, the subretinal space can be created by infusing saline between the photoreceptor and RPE cell layers to create a space for delivery of therapeutic agents. Soluble subretinal medications that are targeted to the subretinal space include tissue plasminogen activator and voretigene neparvovec-ryzl. The former is used to liquefy submacular hemorrhage and the latter for gene therapy of RPE65-associated retinopathy. The alpha-IMS electronic retinal implant, a microphotodiode array to replace the function of lost photoreceptor cells, was also designed for subretinal placement. Cell therapy agents, including human umbilical tissue-derived cells (hUTC) and stem cell-derived RPE cells are also being targeted to the subretinal space.

In one aspect, the presently disclosed subject matter provides system components for performing MISA-based delivery of therapeutic modalities to the subretinal space. Because MISA employs an image-guided ab externo route to the subretinal space, MISA-based delivery of therapeutic modalities to the subretinal space will typically comprise: (1) an imaging component; (2) a Coaxial Guide (COG) component configured to: (a) stabilize the imaging component, e.g., via vacuum suction on scleral surface; and (b) a needle drive to facilitate the separation of the photoreceptor and RPE cell layers, e.g., by injecting fluid between the photoreceptor and RPE layers; and (3) a subretinal access cannula (SAC) component to deliver the therapeutic modality to the desired location within the subretinal space.

In certain embodiments, the imaging component of the MISA-based delivery system is an optical coherence tomography sensor. To increase surgical precision and safety of the MISA approach, certain embodiments of the present disclosure will employ common-path swept-source optical coherence tomography (CP-SSOCT) imaging. CP-SSOCT imaging enables directional, real-time, and depth-resolved A-scan visualization of structures distal to and coaxial with the distal sensor. In certain embodiments, a CP-SSOCT distal sensor is employed to develop MISA according to the principles of image-guided surgery (IGS): to use imaging technology in the operative field to provide real-time information on the precise location of a surgical instrument relative to anatomic structures of interest.

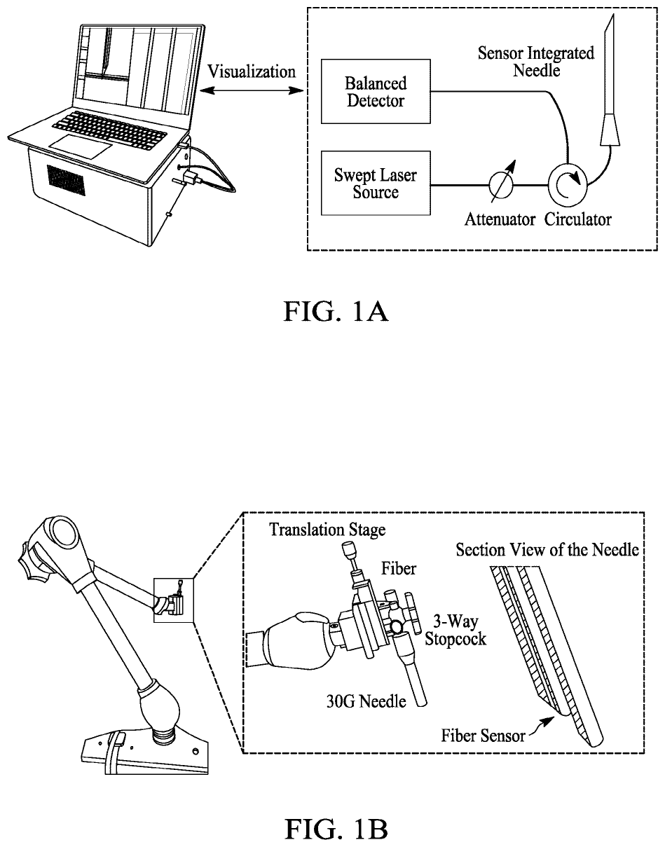

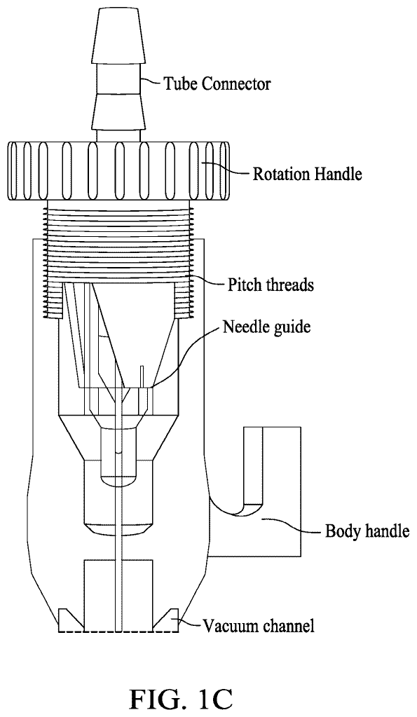

In certain embodiments, a CP-SSOCT device can be employed to acquire real-time depth information of the imaged tissue and the delivery device position. A wide variety of CP-SSOCT devices are suitable for use in connection with MISA-based delivery of therapeutic modalities. For example, but not limitation, such devices can utilize a reference signal. In certain embodiments, the reference signal is from the distal end of a fiber probe. In certain embodiments, the sample and reference beam share the same single-mode fiber, which can limit dispersion and polarization noise. In certain embodiments, the light source is a swept-source OCT engine (AXSUN, Billerica MA USA). In certain embodiments, the light source will have a center wavelength of 1060 nm and a sweeping rate at 100 kHz. In certain embodiments, a broadband circulator (OF-Link, BPICIR-1060-H6) can be used to combine the reference and sample beam to generate an interference signal. In certain embodiments, the spectrum data can be detected by a balanced detector integrated into the OCT engine and collected by a frame grabber (National Instrument, PCI-E-1433). In certain embodiments, the overall system can be packed in a benchtop electronics enclosure. In certain embodiments, the benchtop electronics enclosure is configured as illustrated in FIG. 1A. In certain embodiments, the measured axial resolution of the device can be 4.5 microns inside tissue. In certain embodiments, the scanning range can be 3.7 mm in the air.

In certain embodiments, the CP-SSOCT device is a CP-SSOCT distal-sensor guided injection device. In certain embodiments, a CP-SSOCT distal-sensor guided injection device integrates an optical sensor with a high-index epoxy lens, secured inside a 30-gauge needle with a fixed offset from the tip. In certain embodiments, a three-way stopcock can be used to facilitate an additional port for a syringe to be connected for injection of material. In certain embodiments, the needle and stopcock can be mounted on a translation stage. In certain embodiments, an articulated arm can be used to enhance mobility of the CP-SSOCT distal-sensor guided injection device. For example, but not limitation, the CP-SSOCT distal-sensor guided injection device can be configured as illustrated in FIG. 1B.

In certain embodiments, the MISA-based delivery systems comprise a COG component configured to: (a) stabilize the imaging component, e.g., via vacuum suction on scleral surface; and (b) a needle drive to facilitate the separation of the photoreceptor and RPE cell layers, e.g., by injecting fluid between the photoreceptor and RPE layers. In certain embodiments, the COG can consist of two parts: the eye adaptor and the needle drive.

In cetain embodiments, the eye adaptor of the COG component can be formed by the main body of the COG and can include a vacuum channel to fix the device onto the eye. An exemplary COG component illustrating the configuration of the vacuum channel relative to the main body of the COG is depicted in FIG. 1C. In certain embodiments, the diameter of the vacuum channel is about 10 mm. In certain embodiments, the main body of the COG can comprise a tube connector allowing for: (1) electronic communication to the imaging component sensor, e.g., a fiber sensor, disposed within the COG; and/or (2) delivery of fluid to the needle disposed within the COG. In certain embodiments, the main body of the COG can comprise a body handle to facilitate placement and manipulation of the COG.

In certain embodiments, the needle drive of the COG component can be configured as illustrated in FIG. 1C. For example, but not limitation, the needle drive can comprise a rotation handle, pitch threads, and a needle guide. In certain embodiments, the needle drive can employ pitch threads having a thread pitch of about 500 microns for advancing the needle disposed within the needle guide. In certain embodiments, rotation of the rotation handle can advance the needle disposed within the needle guide forward making use of the pitch threads.

While a variety of suitable COGs can be used in connection with the methods described herein, the exemplary COG depicted in FIG. 1C was 3D-printed by a Stratasys Objet30 Pro PolyJet printer using VeroBlue material. The weight of the exemplary COG guide device depicted in FIG. 1C is 13.09 grams.

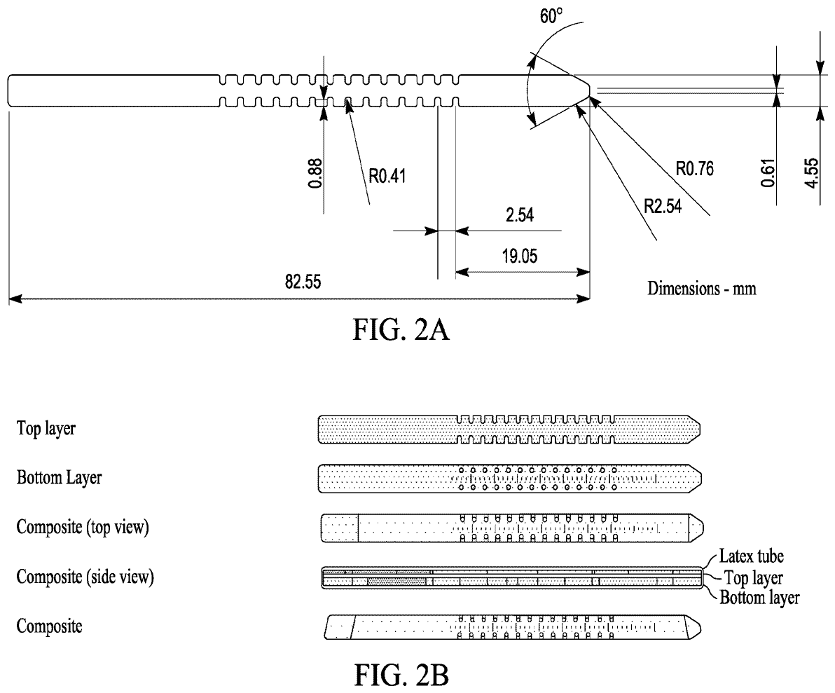

In certain embodiments, the MISA-based delivery systems comprise a SAC component. In certain embodiments, the SAC component can be configured as illustrated in FIG. 2. In certain embodiments, the SAC component can be configured as illustrated PCT application no. PCT/US2019/045074 (WO2020028892), which is incorporated herein by reference in its entirety. For example, but not limitation, the SAC component can be made of top and bottom polyimide layers encased in a latex tube. In certain embodiments, the bottom layer can be about 0.051 mm in thickness. In certain embodiments, marks can be provided at 1 mm intervals beginning at 10 mm from the tip of the bottom layer. In certain embodiments, the top layer can be about 0.025 mm in thickness. In certain embodiments, the latex tube can comprise Mehron liquid latex mixed with distilled water (e.g., in a 3:1 ratio). In certain embodiments, the latex mixture can be degassed and the tubes created by dip coating 3 mm diameter glass tubes which were cured at about 50° C. for about 12 hours. Latex tubes manufactured in this way can then be removed from the 3 mm diameter glass tubes and rinsed with soap. In certain embodiments, the wall thickness was approximately 0.5 mm. In certain embodiments, a composite tube is created by encasing top and bottom polyimide layers in the latex tube. In certain embodiments, the latex tube is configured to start at the shoulder of the tip of the polyimide layers and can extend to 10 mm from the proximal end of the polyimide layers. In certain embodiments, a flexible cyanoacrylate adhesive can be used to adhere the distal ends of the polyimide layers to the latex. In certain embodiments a primer can be used to improve bond quality. Other suitable strategies for adhering the latex tube to the polyimide layers are known in the art and can be used in connection with the methods and compositions described herein.

In another aspect, the presently disclosed subject matter provides MISA-based methods for delivering material, e.g., therapeutic modalities, to the subretinal space.

In certain embodiments, delivery of material, e.g., therapeutic modalities, to the retina can comprise: visualizing the subretinal space using an optical sensor stabilized by a COG comprising a vacuum channel; accessing the subretinal space using a needle disposed in the coaxial guide device; and depositing the material in the subretinal space using a SAC.

In certain embodiments, the optical sensor used in connection with MISA-based methods for delivering material, e.g., therapeutic modalities, to the subretinal space is an optical coherence tomography sensor. In certain embodiments, the optical coherence tomography sensor is a CP-SSOCT device. In certain embodiments, the CP-SSOCT device is a CP-SSOCT distal-sensor guided injection device. In certain embodiments, the optical sensor, e.g., a CP-SSOCT distal-sensor guided injection device, is stabilized by contacting the COG comprising a vacuum channel to a scleral surface.

In certain embodiments, accessing of the subretinal space is achieved via an incision in the sclera and choroid. In certain embodiments, the incision in the sclera and choroid is achieved by advancement of the needle disposed in a needle guide within the COG. For example, but not limitation, the COG can comprise a needle drive comprising a rotation handle, pitch threads, and the needle guide. In certain embodiments, the needle drive can employ pitch threads having a thread pitch of about 500 microns for advancing the needle disposed within the needle guide. In certain embodiments, rotation of the rotation handle can advance the needle disposed within the needle guide forward making use of the pitch threads. In certain embodiments, the methods of the present disclosure comprise introducing a fluid into the subretinal space upon the creation of an incision in the sclera and choroid. In certain embodiments, the fluid is introduced into the subretinal space via the needle. In certain embodiments, introduction of the fluid into the subretinal space creates a bleb. In certain embodiments, the creation of a bleb facilitates the introduction of an SAC into the subretinal space for delivery of the material, e.g., therapeutic modalities.

### Strategies for Accessing Additional Anatomical Spaces

While the present disclosure employs access and delivery to the subretinal space as an exemplary embodiment, the present disclosure is not, however, limited to the access of the subretinal space. For example, the methods and systems described herein relate to IGS approaches to access a variety of additional cavities or natural cleavage planes (i.e., anatomical spaces) in the human body using fiber-optic distal sensor-based imaging, e.g., optical coherence tomography (OCT)-based imaging. For example, the present disclosure is directed, in certain embodiments, to methods and systems for accessing an anatomical space of interest where the anatomical space of interest is selected from: a spinal cord space; an intracranial space, e.g., a ventricle space; a subarachnoid space; a submeningeal space; an inner ear space; a nasopharynx space; an intra-articular space; a lung/pleural space; an intracardiac, and a pancreatic/gastrointestinal space. In certain embodiments, the anatomical space of interest is a spinal cord space. In certain embodiments, the anatomical space of interest is an intracranial space, e.g., a ventricle space. In certain embodiments, the anatomical space of interest is a subaracnoid space. In certain embodiments, the anatomical space of interest is a submeningeal space. In certain embodiments, the anatomical space of interest is an inner ear space. In certain embodiments, the anatomical space of interest is a nasopharynx space. In certain embodiments, the anatomical space of interest is a lung/pleural space. In certain embodiments, the anatomical space of interest is an intracardiac space. In certain embodiments, the anatomical space of interest is a pancreatic/gastrointestinal space.

In certain embodiments, the methods and systems described herein for accessing an anatomical space of interest, where the anatomical space of interest is selected from a spinal cord space; an intracranial space, e.g., a ventricle space; a subarachnoid space; a submeningeal space; an inner ear space; a nasopharynx space; an intra-articular space; a lung/pleural space; an intracardiac, and a pancreatic/gastrointestinal space, comprise a guide device comprising: (1) an adapter configured to contact a surface at least partially encapsulating, directly or indirectly, the anatomical space of interest; and (2) a needle drive comprising a needle disposed in a needle guide. In certain embodiments, the adaptor comprises a vacuum channel. In certain embodiments, the adapter comprises an imaging component. In certain embodiments, the imaging component is stabilized via vacuum suction of the vacuum channel contacting the surface of the anatomical space if interest. In certain embodiments, the needle drive comprises a rotation handle and pitch threads operably connected to the needle guide. In certain embodiments, rotation of the rotation handle advances the needle disposed in the needle guide. In certain embodiments, the imaging component comprises an optical coherence tomography sensor for visualization of an entry point of the needle, such that a user can determine depth of penetration of the needle at entry. In certain embodiments, the optical coherence tomography sensor is positioned within the lumen of the needle

As noted above, the present disclosure is directed, in certain embodiments, to methods and systems for delivering material to an anatomical space of interest other than the subretinal space. For example, but not by way of limitation, the methods and systems for delivering material to such an anatomical space of interest can comprise: visualizing the anatomical space of interest using an optical sensor stabilized by a coaxial guide device comprising a vacuum channel; accessing the anatomical space of interest using a needle disposed in the coaxial guide device; and depositing material in the anatomical space of interest using an access device comprising: a pair of thin, elongate strips of flexible material and thin layer of elastic material surrounding the pair of thin, elongate strips of flexible material form a flexible, expandable tube, wherein an elongate lumen extends between the pair of thin, elongate strips of flexible material for depositing the material. In certain embodiments, the anatomical space of interest is a spinal cord space. In certain embodiments, the anatomical space of interest is an intracranial space, e.g., a ventricle space. In certain embodiments, the anatomical space of interest is a subaracnoid space. In certain embodiments, the anatomical space of interest is a submeningeal space. In certain embodiments, the anatomical space of interest is an inner ear space. In certain embodiments, the anatomical space of interest is a nasopharynx space. In certain embodiments, the anatomical space of interest is a lung/pleural space. In certain embodiments, the anatomical space of interest is an intracardiac space. In certain embodiments, the anatomical space of interest is a pancreatic/gastrointestinal space.

### Exemplary Embodiments

A. In certain non-limiting embodiments, the presently disclosed subject matter provides for a coaxial guide device comprising: an adapter comprising a vacuum channel; and a needle drive comprising a needle disposed in a needle guide.

A1. The foregoing coaxial guide device of A, wherein the vacuum channel is configured to contact a surface of an anatomical space of interest.

A2. The foregoing coaxial guide device of A, wherein the adapter comprises an imaging component.

A3. The foregoing coaxial guide device of A, wherein the imaging component is stabilized via vacuum suction of the vacuum channel contacting the surface of the anatomical space of interest.

A4. The foregoing coaxial guide device of A, wherein the adaptor is an eye adaptor.

A5. The foregoing coaxial guide device of A4, wherein the eye adapter comprises an imaging component.

A6. The foregoing coaxial guide device of A5, wherein the vacuum channel is configured to contact a scleral surface.

A7. The foregoing coaxial guide device of A6, wherein the imaging component is stabilized via vacuum suction of the vacuum channel contacting the scleral surface.

A8. The foregoing coaxial guide device of A-A6, wherein the needle drive comprises a rotation handle and pitch threads operably connected to the needle guide.

A9. The foregoing coaxial guide device of A8, wherein rotation of the rotation handle advances the needle disposed in the needle guide.

A10. The foregoing coaxial guide device of A9, wherein the imaging component comprises an optical coherence tomography sensor for visualization of an entry point of the needle, such that a user can determine depth of penetration of the needle at entry.

A11. The foregoing coaxial guide device of A10, wherein the optical coherence tomography sensor is positioned within the lumen of the needle.

A12. The foregoing coaxial guide device of A1, wherein the anatomical space is selected from the group consisting of: a spinal cord space; an intracranial space; a subarachnoid space; a submeningeal space; an inner ear space; a nasopharynx space; an intra-articular space; a lung/pleural space; an intracardiac, and a pancreatic/gastrointestinal space.

B. In certain non-limiting embodiments, the presently disclosed subject matter provides a method of delivering material to an anatomical space of interest comprising: visualizing the anatomical space of interest using an optical sensor stabilized by a coaxial guide device comprising a vacuum channel; accessing the anatomical space of interest using a needle disposed in the coaxial guide device; and depositing material in the anatomical space of interest using a pair of thin, elongate strips of flexible material and thin layer of elastic material surrounding the pair of thin, elongate strips of flexible material form a flexible, expandable tube, wherein an elongate lumen extends between the pair of thin, elongate strips of flexible material for depositing the material.

B1. The foregoing method of B, wherein the anatomical space of interest is a subretinal space.

B2. The foregoing method of B, wherein the anatomical space of interest is selected from the group consisting of: a spinal cord space; an intracranial space; a subarachnoid space; a submeningeal space; an inner ear space; a nasopharynx space; an intra-articular space; a lung/pleural space; an intracardiac, and a pancreatic/gastrointestinal space.

B3. The foregoing method of B-B2, wherein the optical sensor is an optical coherence tomography sensor.

B4. The foregoing method of B, wherein the optical sensor is stabilized by contacting the coaxial guide device comprising a vacuum channel to a surface of the anatomical space of interest.

B5. The foregoing method of B1, comprising accessing the subretinal space via an incision in the sclera and choroid.

### EXAMPLES

### Materials and Methods

### MISA System Components

The components of the MISA system were the CP-SSOCT device, the coaxial guide (COG), and the subretinal access cannula (SAC).

A CP-SSOCT device was designed to acquire real-time depth information of the imaged tissue and the delivery device position. The system utilizes a reference signal from the distal end of the fiber probe. The sample and reference beam share the same single-mode fiber, thus limiting dispersion and polarization noise. The light source was a swept-source OCT engine (AXSUN, Billerica MA USA) with a center wavelength of 1060 nm and a sweeping rate at 100 kHz. A broadband circulator (OF-Link, BPICIR-1060-H6) was used to combine the reference and sample beam to generate the interference signal. The spectrum data was then detected by a balanced detector integrated into the OCT engine and collected by a frame grabber (National Instrument, PCI-E-1433). The overall system was packed in a benchtop electronics enclosure (FIG. 1A). The measured axial resolution was 4.5 microns inside the tissue and the scanning range was 3.7 mm in the air. A CP-SSOCT distal-sensor guided injection device was created by integrating the optical sensor with a high-index epoxy lens, secured inside a 30-gauge needle with a fixed offset from the tip. A three-way stopcock facilitated an additional port for a syringe to be connected for injection of material. The needle and stopcock were mounted on a translation stage (FIG. 1B). An articulated arm was used to enhance mobility.

The COG was made to stabilize the CP-SSOCT device intraoperatively by vacuum suction on the scleral surface (FIG. 1C). The COG consisted of two parts: the eye adaptor and the needle drive. The eye adaptor formed the main body of the COG and it included a vacuum channel (diameter of 10 mm) to fix the device onto the eye. The needle drive had a thread pitch of 500 microns for advancing the needle. Rotation of the handle advanced the needle forward. The COG was 3D-printed by a Stratasys Objet30 Pro PolyJet printer using VeroBlue material. The weight of the COG guide device was 13.09 grams.

The SAC was made of top and bottom polyimide layers encased in a latex tube (FIG. 2). The bottom layer was 0.051 mm in thickness and marks were provided at 1 mm intervals beginning at 10 mm from the tip. The top layer was 0.025 mm in thickness. The latex tube was Mehron liquid latex mixed with distilled water (3:1 ratio). The latex mixture was degassed and the tubes were created by dip coating 3 mm diameter glass tubes which were cured at 50° C. for 12 hours. The tubes were removed and rinsed with soap. The wall thickness was approximately 0.5 mm. The composite tube was created by encasing top and bottom polyimide layers in the latex tube. The latex tube started at the shoulder of the tip and extended to 10 mm from the proximal end. A flexible cyanoacrylate adhesive was used to adhere the distal ends of the polyimide layers to the latex and a primer was used to improve bond quality.

### Ex Vivo Testing

Fresh bovine eyes were fixed to a globe mount and the CP-SSOCT integrated subretinal 30 g injection needle was positioned perpendicularly to the scleral surface at the ocular equator. A-scans along with AB-mode recordings were acquired as the needle was advanced from outside the scleral surface into the sub-retinal space.

### Animals

Yorkshire pigs (Archer Farms Inc.) weighing 50 pounds were placed under ketamine (20-30 mg/kg)/xylazine (2-3 mg/kg) pre-anesthetic and isoflurane for maintenance with intravenous 5mL/kg/hr lactated ringer solution administered and monitored by a staff veterinarian. Drops of proparacaine 0.5%, tropicamide 1%, and phenylephrine 2.5% were instilled to dilate the pupils. Intraoperative data were captured photographically and the animals were sacrificed postoperatively by intravenous euthanasia.

### Misa Procedure

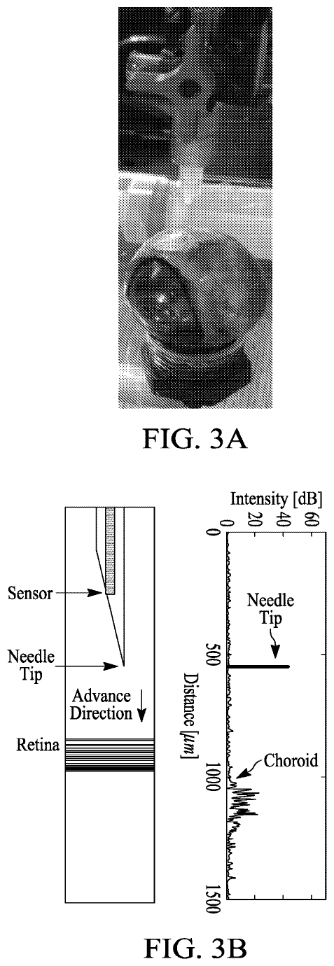

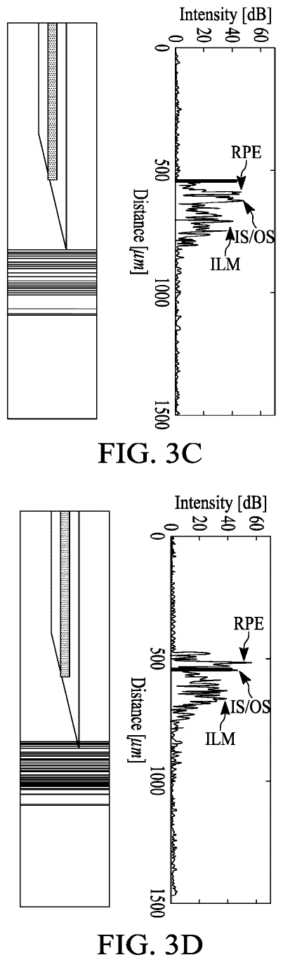

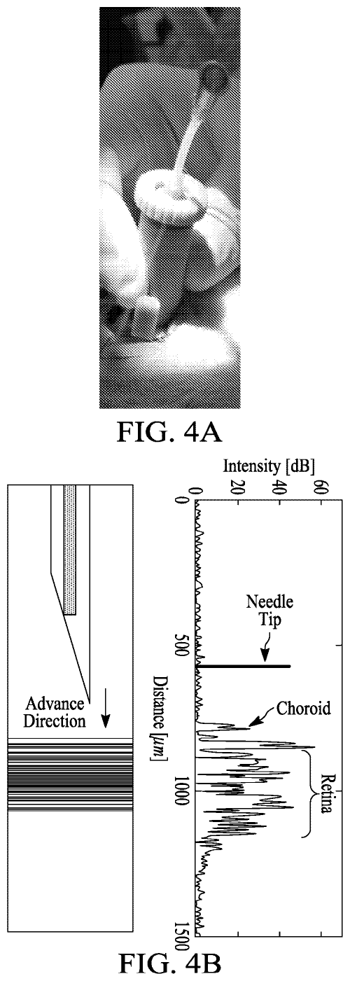

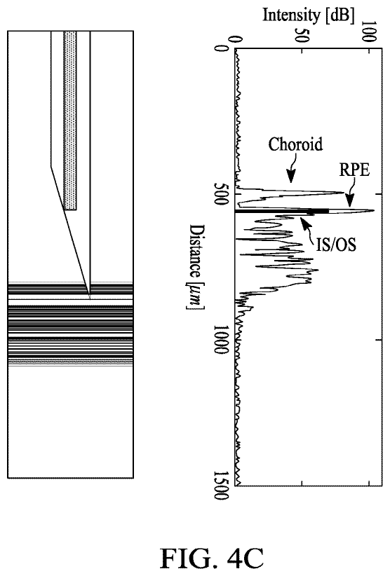

In anesthetized pigs, a corneal traction suture was placed at the inferotemporal corneosleral limbus. To enable intraocular endo-illumination for the purposes of data recording in these pilot experiments, one 27-gauge valved cannulae (Accurus vitrectomy system, Alcon, USA) was placed approximately 4 mm posterior to the limbus for the chandelier (FIG. 3A) and another for balanced salt saline (BSS) infusion. The eye was distracted superonasally. The conjunctiva and Tenon capsule were excised capsule to expose bare sclera. A surgical pen was used to mark the position of the COG. The COG was placed over the exposed sclera and the vacuum seal was actuated (FIG. 3B). The needle drive part of the COG was used to advance a 30-gauge needle (containing the optical sensor) axially towards the eye. By continued actuation of the COG needle drive, the sclera was penetrated by the needle. Real-time CP-SSOCT A- and AB-mode (Quasi-B mode representation from A-scan Images, see FIG. 4) recordings were obtained throughout the process.

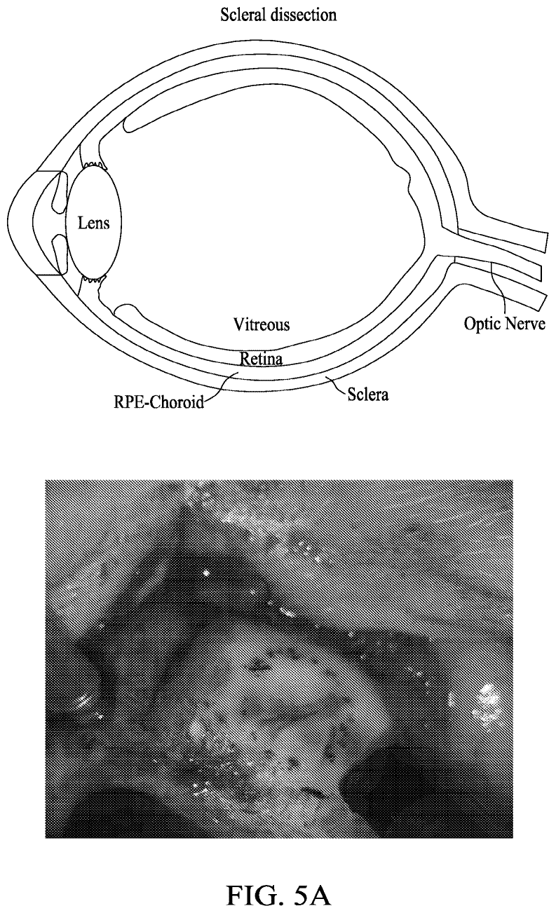

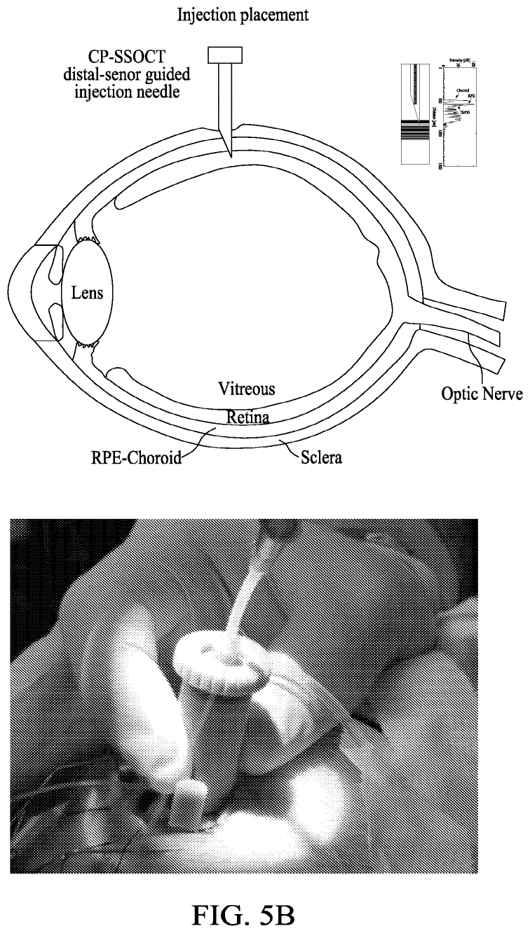

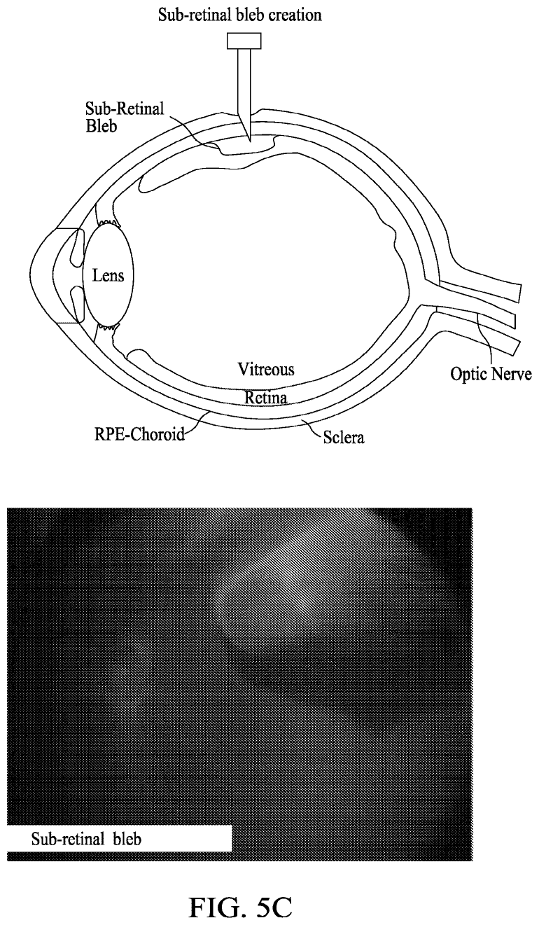

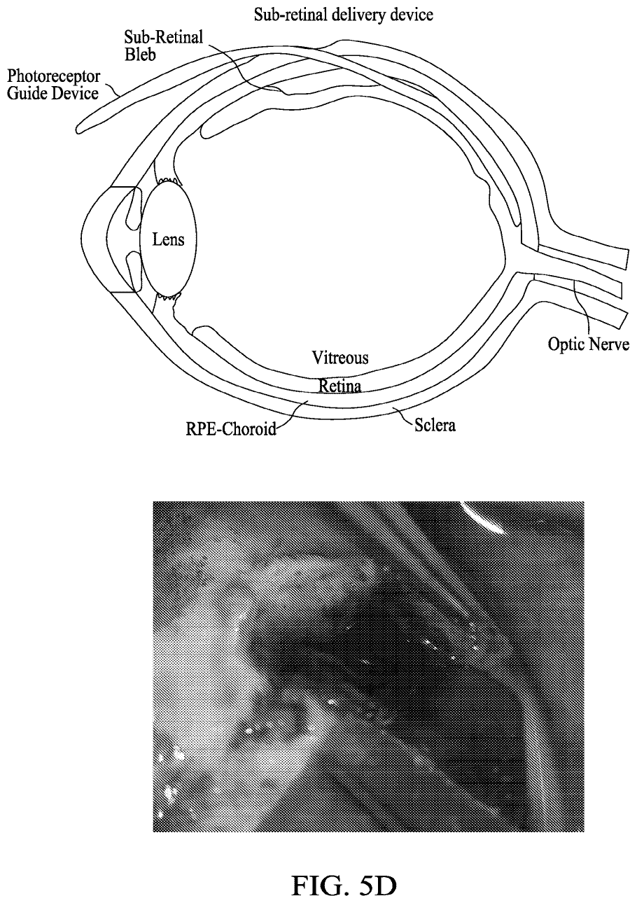

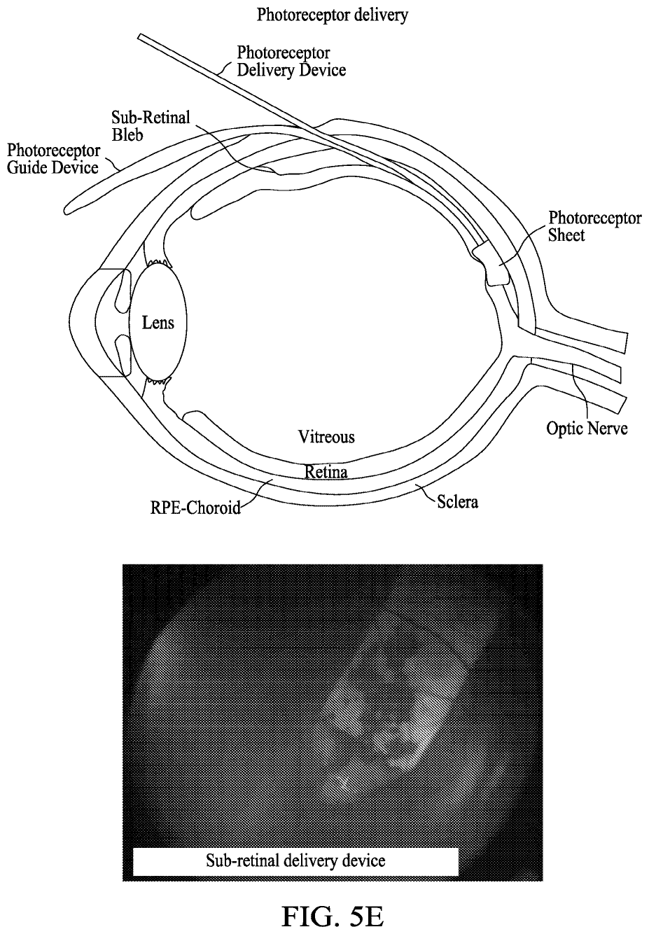

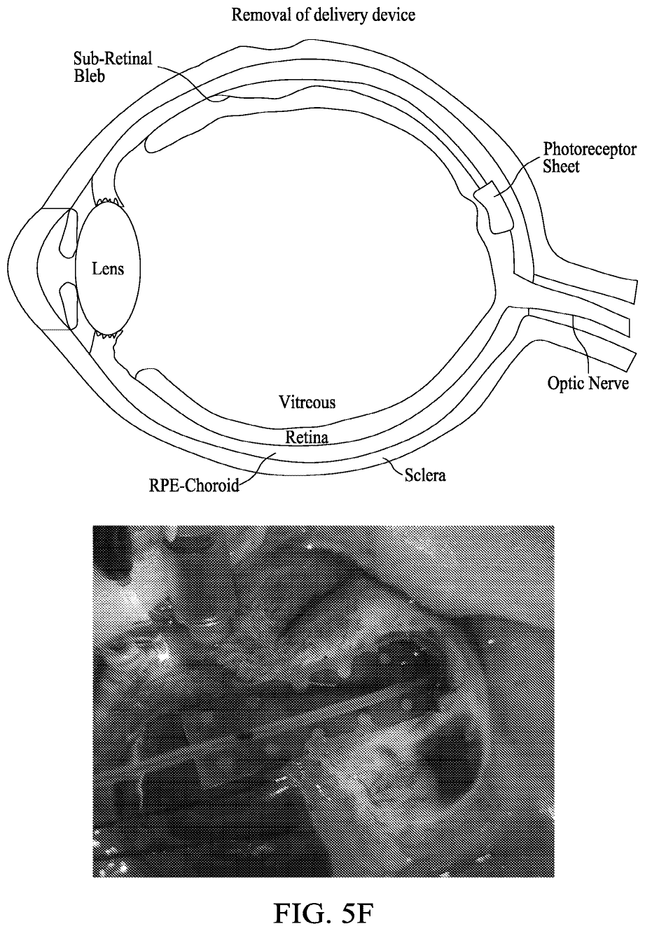

Once the subretinal space was identified by the ab externo CP-SSOCT scan (as shown in FIG. 5), and the needle was adjusted to compensate for the offset of the needle tip and the optical sensor, a subretinal bleb was created by injecting viscoelastic through the syringe port into the subretinal space (FIG. 3C). Subretinal bleb formation was verified by intraocular visualization aided by endo-illumination and scleral indentation when necessary (FIG. 3D).

After subretinal bleb formation was verified, the SAC penetration procedure was initiated. A 5.5 mm-wide sclerotomy was created. The exposed choroid was diathermized to minimize the risk of hemorrhage and then incised with Vannas scissors to create a choroidotomy. The SAC tip was inserted into the sclerotomy and choroidotomy and advanced into the subretinal space (FIG. 3E). The SAC was then manually advanced toward the posterior pole of the eye, propagating inside the subretinal space. The position of the tip of the SAC in the subretinal space of the posterior pole of the eye was verified by intraocular visualization aided by endo-illumination and intraoperative OCT (as shown in FIG. 3F). When the SAC tip was positioned in the desired location at the posterior pole, it was sutured in place to promote stability: the bottom polyimide layer was secured to the limbus with two 7-0 vicryl sutures placed through the premade indentations and holes (shown in FIG. 2). The extraocular portion of the latex sheath was then divided using spring scissors to enable separation of the top and bottom layers of the SAC (FIG. 3G), thus exposing the lumen of the SAC.

### Example 1

### Ex Vivo Validation of Ab Externo CP-SSOCT Retinal Imaging

The ability of the CP-SSOCT device to image the retinal thickness and lamination characteristics from the ab externo side, trans-sclerally, was tested on a bovine eye ex vivo (Figure 4). The sensor integrated needle was positioned perpendicularly to the scleral surface just anterior to the ocular equator as shown in FIG. 4A. A-scan images together with AB-mode images were acquired as the needle was manually advanced into the subretinal space (FIGS. 4B-D). The presumed planar locations of the choroid, RPE, photoreceptor inner and outer segment (IS/OS) line, and the inner limiting membrane (ILM) bands could be identified on the A-scan and AB-mode recordings (FIGS. 4B-D), with reference to the known anatomical features of mammalian retina and the retinal lamination pattern that is typically seen with transpupillary intraocular OCT imaging. Using this system, the bovine peripheral retinal thickness measured approximately 300 μm, which was comparable to that measured by a reference-based intraocular OCT imaging system (data not shown), thus further supporting the interpretation of the tissue layer correlations of the A-scan envelope. The presumed subretinal space was identified as the space corresponding to the hyporeflective band situated between the relatively hyper-reflective RPE and IS/OS bands (FIG. 4D).

### Example 2

### Preliminary Misa Procedure Design

The first two surgeries (n=2 eyes of two pigs) served to acquaint the surgical, imaging, and bioengineering teams with the relevant technical constraints and to arrive at consensus priorities for MISA device and procedure design. The presumptive steps and incisions of the planned surgical procedure were piloted, culminating in attempts to insert passive plastic and metal strips of varying stiffness and thickness into the subretinal space. During these first attempts, significant insight was gained into improvements that could enhance the surgical procedure. Also, the feasibility of passively navigating within the space was confirmed. The approximate geometry and bending stiffness for a passive device was established, with reference to the stiffness and thickness standards that were tested. The feasibility of non-hemorrhagic choroidotomy measuring 5.0 mm-6.0 mm in width, using adequate pre-incision diathermy, was established. The overall maximal width of the device for preclinical testing in swine was set at 5.0 mm. In these first two pigs, a partial thickness limbal-based scleral flap was elevated with a beaver blade at the proposed site of the sclerotomy, to allow for closure akin to that of a trabeculectomy procedure. However, the scleral flap was deemed unnecessary for the remainder of the project.

### Example 3: Preliminary Sac Testing

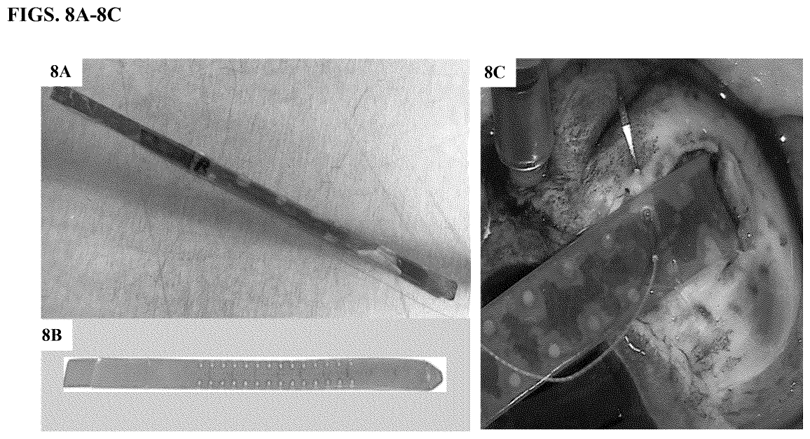

The next surgery (n=1 eye) focused on the specifications of the SAC. The intent of the SAC was to create a smooth-gliding passageway for therapeutic material to be inserted into the subretinal space. For the purposes of this project, the envisioned therapeutic material was a construct of the approximate size, shape, and stiffness of a preformed sheet of cells-with-scaffold similar to those used in several recent clinical trials (Kashani et al., A bioengineered retinal pigment epithelial monolayer for advanced, dry age-related macular degeneration. Sci Transl Med, 2018. 10 (435)). Therefore, the SAC lumen had to be at least 5.00 mm wide. This was done to reduce the scleral thickness of imaging and needle penetration. Several issues were identified regarding SAC geometry. Most notably, the geometry of the tip was too square (FIG. 8A), with the sharp corners prohibiting smooth tip insertion into the sclerotomy. Also, once inside the eye, in some cases the device shifted side-to-side in the subretinal space when the eye was rotated into the primary position to view the location of the device tip through the pupil. Based on these observations, we identified the following improvements: (1) refining the SAC tip to have angled/rounded tip and (2) including “belt holes” to allow for the SAC to be sutured to the sclera or corneoscleral limbus in at least two or more locations.

### Example 4

### Iteration of Sac Design

The SAC tip was modified: the tip was angled and rounded as shown in (FIG. 8B). The tip consisted of 0.025 and 0.051 mm layers of polyimide with the same tip profile. Two rows of holes were added along the length of the device to allow fixation to the sclera and/or cornea next to the insertion site (FIG. 8C). To further smoothen the overall profile of the SAC tip, the latex sheath was cut and adhered so that it ended just prior to the tapered section of the distal end. This was done to reduce roughness at the tip that had caused bleeding in prior surgeries. The top sheet's holes were cut into slots that mirrored the holes on the bottom. This eased fixation of the device and allowed the sutures to be made without cutting back the top layer (FIG. 8C). Rather, the sutures can be made through the latex and the bottom holes. The extraocular portion of the top layer could then be peeled away, after dividing the latex sheath with scissors, to access the lumen. A series of marks were cut into the bottom layer to so that the inserted length could be measured.

### Example 5

### In Vivo Testing of Misa

The iterated prototype MISA system components and procedure, including the CP-SSOCT device, coupled with the COG and SAC components, was then tested on porcine eyes (n=4) in vivo (FIG. 5).

A- and AB-mode images were acquired as the CP-SSOCT sensor integrated needle was advanced in the COG towards and into the subretinal space, through the intact sclera (FIGS. 5A-B). Hyper-reflective signals presumed to correspond with the laminar depths of the choroid, RPE, and IS/OS layer were identified on the A- and AB-mode recordings. The subretinal space was identified as the relatively hyporeflective lamina between the RPE and IS/OS laminae (FIG. 5B). The CP-SSOCT subretinal injection device was capable of creating a subretinal bleb to gain access to the subretinal space through a transscleral approach. The 5.5 mm sclerotomy and equivalent choroidotomy (after diathermy) incisions were created. The SAC was then introduced into the sclerotomy and choroidotomy and then axially propagated manually towards the posterior pole. When the desired position of the SAC tip in the subretinal space at the posterior pole was achieved, for maximal intraoperative stability, fixation of the SAC to the corneoscleral limbus and post-limbal sclera was accomplished using four sutures in the sclera.

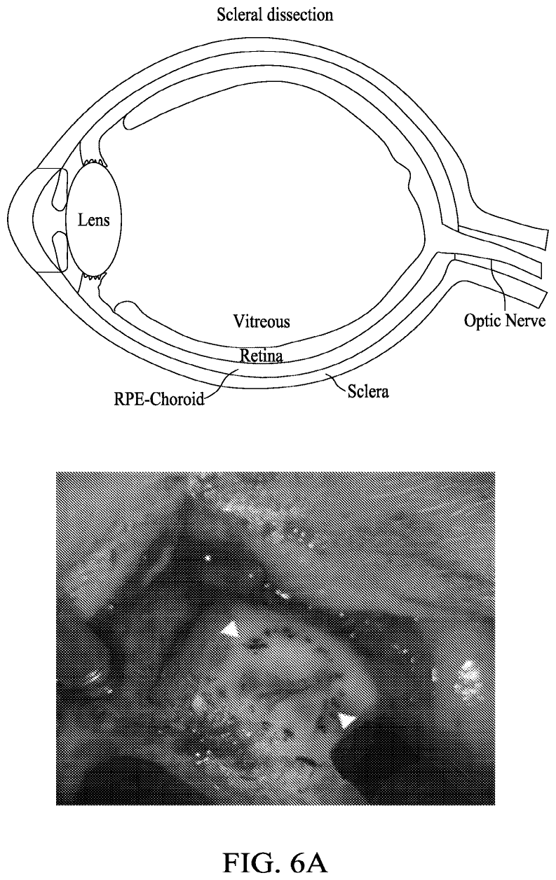

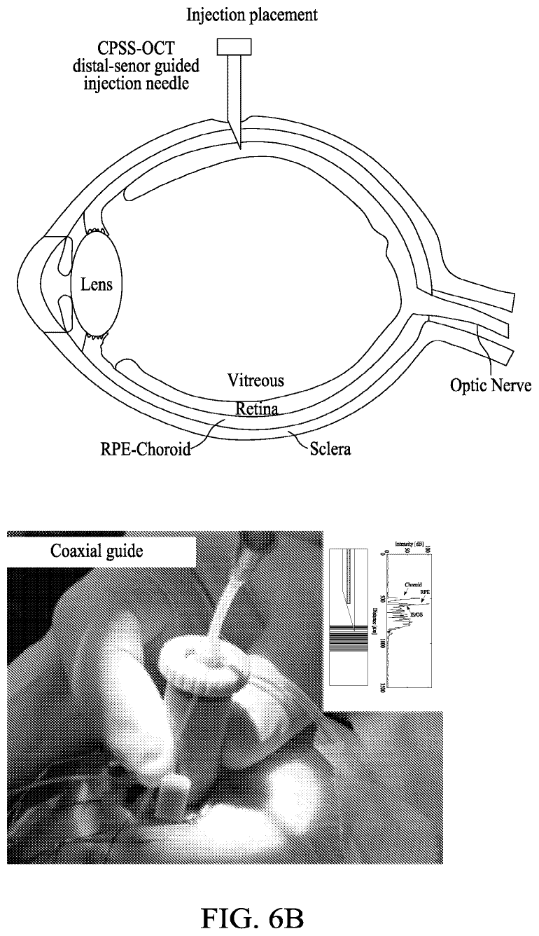

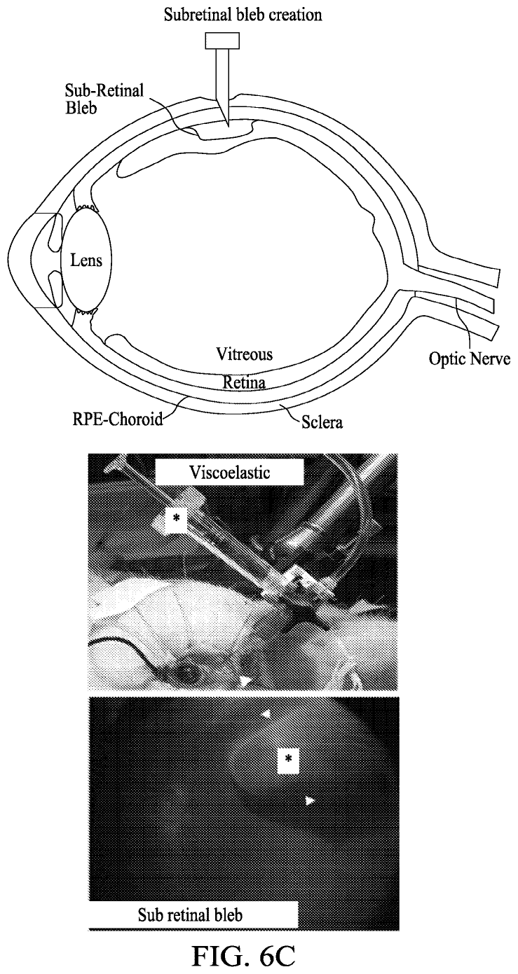

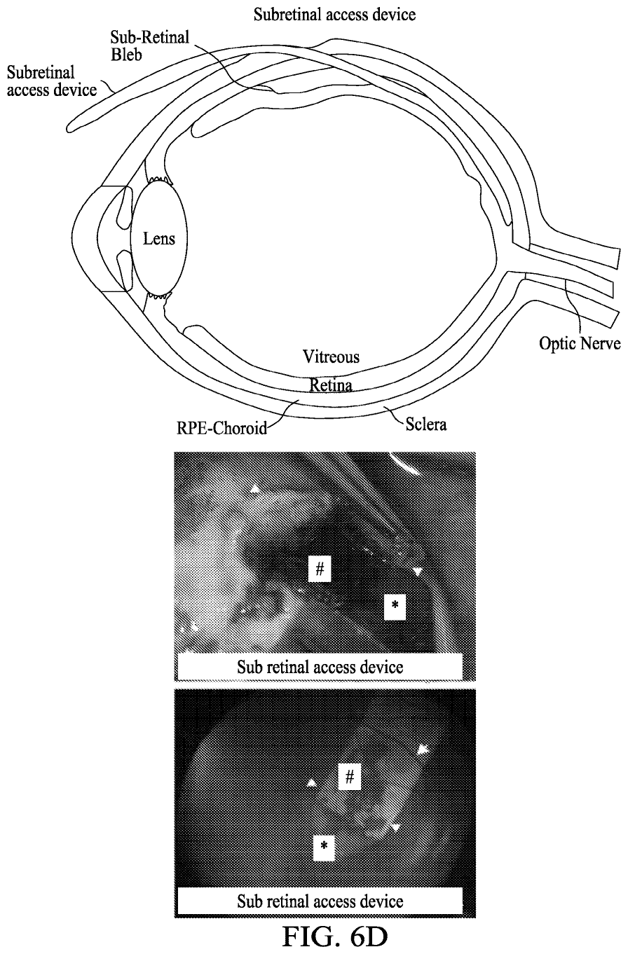

The SAC tip was able to be positioned in the foveal subretinal space in four eyes. The procedural steps summarized in FIG. 6A-6D. Briefly, (6A) the sclera is dissected at the equator and a ring is marked to prepare the sclera for placement of the CP-SSOCT guide device (white arrowhead denote scleral marking edges). (6B) The CP-SSOCT guide device is secured on the sclera, the injection needle is advanced axially, and subretinal space is identified on A-scan (between the RPE and IS/OS A-scan peaks). (6C) The subretinal bleb created by injection of viscoelastic material (top, asterisk—viscoelastic) (arrowhead—subretinal needle) and is visualized internally (bottom, asterisk—subretinal bleb) (arrow heads—bleb borders). (6D) A sub retinal delivery device (top and bottom, asterisk) is inserted into the bleb (top, hashtag—bleb) (top, arrow heads—sclerotomy edges) at the equator, advanced to the posterior retina and confirmed to be in the posterior sub retinal space (bottom, arrow—retinal vessels overlying posterior sub retinal delivery device) (bottom, hashtag-RPE displacement overlying sub retinal delivery device). In the last of the four eyes, intraoperative transpupillary OCT (Leica Proveo 8 with EnFocus OCT) imaging of the posterior pole was performed to verify subretinal placement of the SAC tip. The SAC tip was visualized to be positioned in the subretinal space without obvious macroscopic injury to the overlying retina.

### Example 6

### Complications and Mitigating Strategies

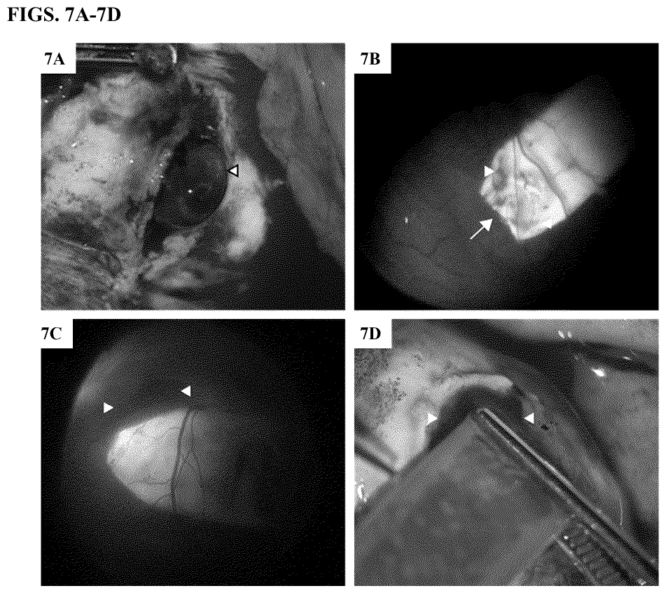

Complications were classified as major or minor depending on the potential risk of causing severe vision loss. Major complications that occurred were retinal incarceration (n=2) (FIG. 7A) and retinal perforation (n=3). Minor complications were RPE displacement (n=4) (FIG. 7B), mild vitreous hemorrhage (n=3), mild subretinal hemorrhage (n=1) (FIG. 7C) and moderate suprachoroidal hemorrhage that egressed out of the eye without causing a hemorrhagic choroidal detachment (n=1) (FIG. 7D).

Retinal incarceration in the sclerotomy occurred in the first animal, when intraocular infusion was maintained with the intraocular pressure set 25 mmHg during the choroidal incision step. Thus, when the choroid was incised, the injected viscoelastic was expelled and the retina externalized, incarcerated, and ruptured through the sclerotomy/choroidotomy. To mitigate against this risk, the procedure was modified in the next animal such that the infusion flow was reduced (intraocular pressure set at 5 mmHg), however the retina still appeared to bulge outwards upon choroidal incision (without rupture, FIG. 7A), thus impeding the insertion of the SAC. For all subsequent animals, the infusion was turned off and prior to the choroidotomy step, and the retina no longer bulged out of the eye, there was no retinal incarceration nor perforation, and the viscoelastic was not expelled.

Vitreous hemorrhage occurred during SAC propagation, presumably because of mechanical stretching of retinal blood vessels by the SAC tip as the vector of SAC propagation was not perfectly parallel to the plane of the subretinal space. The procedure was modified to ensure that the vector of propagation was angled parallel to the subretinal plane as far as possible, and this adjustment appeared to promote the safe propagation of the device until its tip reached the posterior pole of the pig eye, without vitreous hemorrhage, in subsequent animals. RPE displacement was observed wherein patches of pigmented RPE cells were observed to lie superficial to the device as shown in FIG. 7B, instead of beneath it as intended. All publications, patents and other references cited herein are incorporated by reference in their entirety into the present disclosure.

What is claimed is:

1. A coaxial guide device comprising:
an adapter comprising a vacuum channel; and
a needle drive comprising a needle disposed in a needle guide.

2. The device of claim 1, wherein the vacuum channel is configured to contact a surface of an anatomical space of interest.

3. The device of claim 1, wherein the adapter comprises an imaging component.

4. The device of claim 3, wherein the imaging component is stabilized via vacuum suction of the vacuum channel contacting the surface of the anatomical space of interest.

5. The device of claim 1, wherein the adaptor is an eye adaptor.

6. The device of claim 5, wherein the eye adapter comprises an imaging component.

7. The device of claim 6, wherein the vacuum channel is configured to contact a scleral surface.

8. The device of claim 7, wherein the imaging component is stabilized via vacuum suction of the vacuum channel contacting the scleral surface.

9. The device of claim 1, wherein the needle drive comprises a rotation handle and pitch threads operably connected to the needle guide.

10. The device of claim 9, wherein rotation of the rotation handle advances the needle disposed in the needle guide.

11. The device of claim 3, wherein the imaging component comprises an optical coherence tomography sensor for visualization of an entry point of the needle, such that a user can determine depth of penetration of the needle at entry.

12. The device of claim 11, wherein the optical coherence tomography sensor is positioned within the lumen of the needle.

13. The device of claim 2, wherein the anatomical space of interest is selected from the group consisting of: a spinal cord space; an intracranial space; a subarachnoid space; a submeningeal space; an inner ear space; a nasopharynx space; an intra-articular space; a lung/pleural space; an intracardiac, and a pancreatic/gastrointestinal space.

14. A method of delivering material to an anatomical space of interest comprising:
visualizing the anatomical space of interest using an optical sensor stabilized by a coaxial guide device comprising a vacuum channel;
accessing the anatomical space of interest using a needle disposed in the coaxial guide device; and
depositing material in the anatomical space of interest using a pair of thin, elongate strips of flexible material and thin layer of elastic material surrounding the pair of thin, elongate strips of flexible material form a flexible, expandable tube, wherein an elongate lumen extends between the pair of thin, elongate strips of flexible material for depositing the material.

15. The method of claim 14, wherein the anatomical space of interest is a subretinal space.

16. The method of claim 14, wherein the anatomical space of interest is selected from the group consisting of: a spinal cord space; an intracranial space; a subarachnoid space; a submeningeal space; an inner ear space; a nasopharynx space; an intra-articular space; a lung/pleural space; an intracardiac, and a pancreatic/gastrointestinal space.

17. The method of claim 14, wherein the optical sensor is an optical coherence tomography sensor.

18. The method of claim 14, wherein the optical sensor is stabilized by contacting the coaxial guide device comprising a vacuum channel to a surface of the anatomical space of interest.

19. The method of claim 15, comprising accessing the subretinal space via an incision in the sclera and choroid.
